(19) United States
(12) Patent Application Publication
(10) Pub. No.: US 2021/0318301 A1
(43) Pub. Date: Oct. 14, 2021

(54) INTEGRATED IMMUNODIAGNOSTIC FLUORESCENCE READER HAVING MULTIPLE DIAGNOSES FUNCTION

(71) Applicant: BODITECH MED INC., Chuncheon-si, Gangwon-do (KR)

(72) Inventors: Kie Bong NAHM, Seoul (KR); Min Seok CHA, Chuncheon-si (KR); Chu Hyun CHO, Chuncheon-si (KR); Sang Hyun PARK, Seoul (KR)

(21) Appl. No.: 17/285,606

(22) PCT Filed: Nov. 8, 2019
(86) PCT No.: PCT/KR2019/015154

§ 371 Date: Apr. 15, 2021

(30) Foreign Application Priority Data
Nov. 14, 2018 (KR) 10-2018-0139578

(51) Int. Cl. G01N 33/543; G01N 21/64

(52) U.S. Cl. CPC G01N 33/54386; G01N 2201/0221; G01N 2021/6463; G01N 21/6486

(57) ABSTRACT

The present disclosure relates to an integrated immunodiagnostic fluorescence reader having a multiple diagnoses function, the reader being configured to identify and classify samples through barcodes that are allocated differently by diagnosis marker and adjust an optimum reaction time in a lateral flow method, and the present disclosure utilizes barcode information formed on a surface thereof that is the same as that of a fluorometric window of a diagnostic cartridge, so as to identify barcodes even if different kinds of diagnostic cartridges are inserted, thereby preventing errors, and, when a sample is loaded, senses a sample flowing to a development film so as to automatically calculate reaction time, and senses fluorescent information measured through the fluorometric window, thereby enabling simultaneous analysis of diagnostic markers or types of samples and the like.

### TECHNICAL FIELD

The present disclosure relates to a fluorescence reader used for immunodiagnosis, and more particularly, to a fluorescence reader with a multi-diagnosis function, which is configured to be capable of identifying and classifying various samples through barcodes differently assigned for each diagnosis marker and of adjusting an optimal time required for a later flow of samples.

### BACKGROUND ART

Various diagnostic kits for detecting a specific target material in liquid samples, such as blood, have been developed and conveniently and widely used for rapid diagnosis of a disease. Among these kits, a diagnostic kit based on immunochromatographic assay is widely used to detect the status of disease or to monitor its development. In particular, a representative example is a test based on a lateral flow method.

In such diagnostic kits, a fluorescent material is used as a labeling agent for detecting a target molecule and accordingly, a reader for detecting the signal from the fluorescent material is required. Although such fluorescence readers may be able to perform an accurate detection per se, its complicated structure may result in malfunctions and thus inaccurate results even by the trained user in many cases.

In addition, recently there are emerging multi-diagnostic type devices having high sensitivity and being capable of perform various types of tests compared to a rapid kit which depends on the human eye for reading the results. However, such multi-diagnostic type devices tend to become more complicated to use and complex in structure than a single test device and to have a size larger than the single test device. Also is a problem a high production cost associated with a longer time for manufacturing and quality control.

In a lateral flow method for quantification, one of the factors to increase the accuracy is to scan a signal from a cartridge at an exact predetermined time. In a conventional method, in order to save time to obtain the results from the test using multiple cartridges, a semiautomatic process is used in which samples are loaded into a cartridge outside of a fluorescence reader, and incubated for a certain amount time for reaction to occur, then the cartridge is inserted into the reader, and the button is pressed to start a scan. A problem of this process is that the reaction time may not be optimal as manufacturers intend. The reason for this is that a total measurement time may unintentionally vary and thus be increased or decreased depending on various factors such as the time taken for a user to insert the cartridge at the end of a reaction time, the time taken for the user to press the button, and the time taken for the reader to perform a scan, which may be different case by case or user by user basis. If the total measurement time is increased as expected, the test may become inefficient, or if the total measurement time is decreased, the quality of the test is not optimal. Accordingly, there is a need for development a fluorescence reader capable of overcoming such disadvantages.

Furthermore, in a multi-diagnostic device for diagnosing or testing several types of diseases not a device for diagnosing a single disease, it is also necessary to implement an error prevention mechanism capable of preventing a danger which may occur when a user mistakenly inserts a wrong cartridge for test.

Korean Patent Application Publication No. 10-2015-0029290 relates to a device of a reader for diagnostic strip, and discloses a reader characterized by comprising a stage on which a diagnostic strip having two or more detection areas lengthwise is located, a light source and a light detector disposed on one side of the stage, and means for movement for moving the stage lengthwise, wherein signal can be continuously obtained from the two or more detection areas (reaction area and comparative area) by moving the diagnostic strip by the movement means. However, the device has problems in that a user cannot perform a measurement at an exact time intended and mixing of the cartridges cannot be prevented when the multiple cartridges of different samples are employed.

### SUMMARY OF THE INVENTION

Aspects of the present invention provide a fluorescence reader used for immunodiagnosis having a multi-diagnosis function and an error prevention function, which can prevent an error by identifying different types of diagnostic cartridges when various diagnostic cartridges are inserted. Aspects of the present invention can simultaneously identify the different type of diagnosis markers or samples, etc. by automatically calculating a reaction time by detecting a sample flowing into a membrane when the sample is loaded.

One aspect the present invention provides a fluorescence reader which may be used for immunodiagnosis, comprising: a base frame with an open top surface, comprising: an entrance part at a front side thereof into which a diagnostic cartridge having a barcode differently assigned to each diagnosis marker and a fluorescence measurement window is inserted wherein the barcode is located on the same side as the fluorescence measurement window; an inner space opened to an outside through the entrance part, wherein the cartridge inserted through the entrance part; a space for the movement of an optical module, being an upper part of the inner space in which an optical module moves along two guide shafts each fixed to the base frame in parallel to a lengthwise direction of the diagnostic cartridge located in the inner space; a space for driving module mounting, being located on one side of the space for the movement of the optical module; a space for reference position detection module mounting wherein the detection module detects a reference position of the optical module; a cartridge fixing part fixing the diagnostic cartridge at a position by applying pressure at least part of the diagnostic cartridge when the diagnostic cartridge is inserted into the inner space; fitting grooves into each of which one end of each of the two guide shafts is fit and fixed and mounting grooves into each of which the other end of each of the two guide shafts is mounted; an optical module located in the space for the movement of an optical module of the base frame and configured to radiate light to the fluorescence measurement window and barcode of the diagnostic cartridge while moving in parallel along the two guide shafts to detect fluorescence light and ambient light reflected by the radiated light; a driving module located in the space for driving module mounting of the base frame and connected to the optical module to move the optical module in parallel; a detection module for a reference position located in the space for reference position detection module mounting of the base frame and configured to detect a reference position of the optical module and to detect whether the cartridge is inserted; and an upper frame 150 configured to cover and fix the opened top surface of the base frame comprising a guide shaft fixing protrusion fixing one end of each of the guide shafts together with the guide shaft mounting groove of the base frame and a fastening part fastened to the base frame in order to maintain the fixing by the guide shaft fixing protrusion.

One aspect the present invention provides a fluorescence reader for immunodiagnosis, wherein the optical module comprises: a module casing comprising a module lower part and a module upper plate covering and fixing an opened top surface of the module lower part to form an box shaped internal space; a light source disposed within the module casing; an ambient light guide part disposed within the module casing and configured to guide a light from the light source to the barcode of the diagnostic cartridge and to guide an ambient light reflected from the barcode to a light detector; a fluorescence guide part disposed within the module casing comprising a dichroic mirror for guiding a light from the light source to the fluorescence measurement window of the diagnostic cartridge and for guiding a fluorescence emitted from the fluorescence measurement window to a light detector; and a light detector configured to detect the ambient light and the fluorescence; wherein the module lower part on one side thereof has a cylinder barrel into which one of the two guide shafts is put in to guide the optical module in a lengthwise direction, and on the other side thereof has guide protrusions to guide the optical module along the other of the two guide shafts, and on the bottom side thereof has an interrupt protrusion part being inserted into the detection module for a reference position to determine the reference position.

One aspect the present invention provides a fluorescence reader for immunodiagnosis, wherein the fluorescence guide part comprises: a lens for light source for concentrating irradiation light emitted from the light source in a horizontal lateral direction; a dichroic first mirror for changing a path of the irradiation light concentrated by the lens for light source so that the irradiation light travels in a horizontal and front direction, and for transmitting straight the fluorescence emitted from the fluorescence measurement window of the diagnostic cartridge; a second mirror for changing a path of the light whose path has been changed by the dichroic first mirror in a vertical and downward direction to send the light to the fluorescence measurement window of the diagnostic cartridge, and for sending the fluorescence to the first mirror, generated from the fluorescence measurement window of the diagnostic cartridge; a downward lens for concentrating the light whose path has been changed by the second mirror, irradiating the concentrated light to the fluorescence measurement window of the diagnostic cartridge, concentrating the fluorescence emitted from the fluorescence measurement window, and sending the concentrated fluorescence to the second mirror; a detection lens 254 for concentrating the fluorescence whose path has been changed by the second mirror and transmitted straight the first mirror, and concentrated by the downward lens; and a pin hole 256 for filtering, transmitting and guiding the fluorescence concentrated by the detection lens.

One aspect the present invention provides a fluorescence reader for immunodiagnosis, wherein the light source 220 is one, wherein the ambient light guide part shares a path of light incident on the barcode with the path of the fluorescence guide part, wherein further comprises a mirror for adjusting the path of light incident on the barcode or a path of light reflected from the barcode so that the ambient light reflected from the barcode is perpendicularly incident on the light detector.

One aspect the present invention provides a fluorescence reader for immunodiagnosis, wherein the light detector comprises an ambient light detector and a fluorescence detector, wherein the ambient light detector is disposed on the path of the light emitted from the barcode so that the ambient light reflected from the barcode is perpendicularly incident on the ambient light detector.

One aspect the present invention provides a fluorescence reader for immunodiagnosis, wherein the light sources are two, wherein the path of the light incident on the barcode is different in the ambient light guide part and in the fluorescence guide part, wherein each light emitted by the two light sources is guided to the detectors along the ambient light guide part and along the fluorescence guide part, respectively, wherein a light source of the light incident on the ambient light guide part is disposed adjacent to the light detector so that the ambient light reflected from the barcode is perpendicularly incident on the light detector.

One aspect the present invention provides a fluorescence reader for immunodiagnosis, wherein the light detector comprises the ambient light detector and the fluorescence detector, wherein the light source of the light incident on the ambient light guide part and the ambient light detector are adjacently disposed so that the ambient light reflected from the barcode is perpendicularly incident on the light detector.

One aspect the present invention provides a fluorescence reader for immunodiagnosis, wherein the driving module comprises: a motor configured to provide a rotatory power; two guide shafts each having one end fixed to the fitting grooves of the base frame and the other end mounted in the mounting grooves, disposed in parallel to the lengthwise direction of the diagnostic cartridge inserted into the inner space, and the two guide shafts configured to guide the optical module by the cylinder barrel and guide protrusions and; a driving shaft 320 connected to the motor in a way to rotate and disposed on at least one lateral side of the optical module, and extending in a front and back direction; and a driving carrier disposed between the optical module and the driving shaft and connected to the optical module and the driving shaft, wherein when the motor rotates and thus the driving shaft is rotated, the driving carrier is displaced in the front and back direction, and the optical module connected to the driving carrier is guided by the two guide shafts and displaced in the front and back direction.

One aspect the present invention provides a fluorescence reader for immunodiagnosis, wherein the cartridge fixing part of the base frame comprises: a fixing spring configured to lift up and fix a bottom of the diagnostic cartridge; a lifting-up protrusion configured to be matched with and fix a portion of the bottom groove of the diagnostic cartridge; and a side fitting frame 134 configured to fix the diagnostic cartridge by inserting a side of the diagnostic cartridge therein.

One aspect the present invention provides a fluorescence reader for immunodiagnosis, wherein the reference position detection module comprises: a module main board fixed in a direction opposite to the entrance part of the base frame; and an interrupt sensor part disposed in a direction opposite to the entrance part on the module main board, wherein the interrupt protrusion part is inserted into the interrupt sensor part, wherein a position of the optical module is displaced between the reference position where the interrupt protrusion part is inserted into the interrupt sensor part and a position of the entrance part.

One aspect the present invention provides a fluorescence reader for immunodiagnosis, wherein the reference position detection module further comprises a cartridge sensing switch disposed under the module main board, wherein the diagnostic cartridge is configured to press the cartridge sensing switch when the diagnostic cartridge is inserted into the base frame through the entrance part and an end of the diagnostic cartridge reaches a specific position, wherein the cartridge sensing switch generates an insertion signal for the diagnostic cartridge when the diagnostic cartridge presses the cartridge sensing switch.

One aspect the present invention provides a fluorescence reader for immunodiagnosis, wherein the diagnostic cartridge comprises the fluorescence measurement window extended in a lateral flow direction of a sample and having the lateral flow of the sample exposed upward, and a barcode for identifying a type of sample, wherein the optical module is configured to: identify the type of sample by reading the barcode by moving to the barcode area, move to an end point of the fluorescence measurement window after detecting lateral flow start time of the sample at a start point of the fluorescence measurement window, and calculate a flow rate of the lateral flow by detecting lateral flow end time of the sample at the end point of the fluorescence measurement window.

One aspect the present invention provides a fluorescence reader for immunodiagnosis, wherein the diagnostic cartridge comprises the fluorescence measurement window extended in a lateral flow direction of a sample and having the lateral flow of the sample exposed upward, and a barcode, wherein the optical module is configured to: identify the type of sample by reading the barcode by moving for the barcode light source to irradiate a light to the barcode; move to an end point of the fluorescence measurement window after detecting lateral flow start time of the sample at a start point of the fluorescence measurement window, and calculate a flow rate of the lateral flow by detecting lateral flow end time of the sample at the end point of the fluorescence measurement window.

One aspect the present invention provides An immunodiagnostic fluorescence reader according to the present disclosure can prevent an error by identifying barcode although different types of diagnostic cartridges are inserted using barcode information formed on the same surface as the fluorescence measurement window of the diagnostic cartridge, can automatically calculate a reaction time by detecting a sample flowing into a membrane when the sample is loaded, and can simultaneously analyze the type of diagnosis marker or sample by detecting fluorescent information measured through the fluorescence measurement window.

### DESCRIPTION OF DRAWINGS

FIGS. 1A and 1B are diagrams illustrating an integrated immunodiagnostic fluorescence reader having multiple diagnosis function according to the present disclosure.

FIGS. 2A and 2B are diagrams illustrating an optical module of the integrated immunodiagnostic fluorescence reader having multiple diagnosis function according to the present disclosure.

FIGS. 3A-3D are diagrams illustrating parts of the optical module in detail.

FIGS. 4A-4D are diagrams illustrating a diagnostic cartridge of the integrated immunodiagnostic fluorescence reader having multiple diagnosis function according to the present disclosure.

FIGS. 5A and 5B are diagrams illustrating the optical module and a driving module.

FIGS. 6A and 6B are diagrams illustrating a position detection module of the integrated immunodiagnostic fluorescence reader having multiple diagnosis function according to the present disclosure.

FIG. 7 is a diagram illustrating one cross section of the integrated immunodiagnostic fluorescence reader having multiple diagnosis function according to the present disclosure on which the optical module and the driving module are mounted.

FIGS. 8A-8D are plan views illustrating a base frame of the integrated immunodiagnostic fluorescence reader having multiple diagnosis function according to the present disclosure.

FIGS. 9A and 9B are diagrams illustrating a structure in which a guide shaft is fixed by a base frame and an upper frame.

FIGS. 10A and 10B are diagrams illustrating part of a cross section of the integrated fluorescence reader for multi-diagnosis on which the diagnostic cartridge is mounted.

### DETAILED DESCRIPTION OF EMBODIMENTS

Hereinafter, preferred embodiments according to the present disclosure are described with reference to the accompanying drawings.

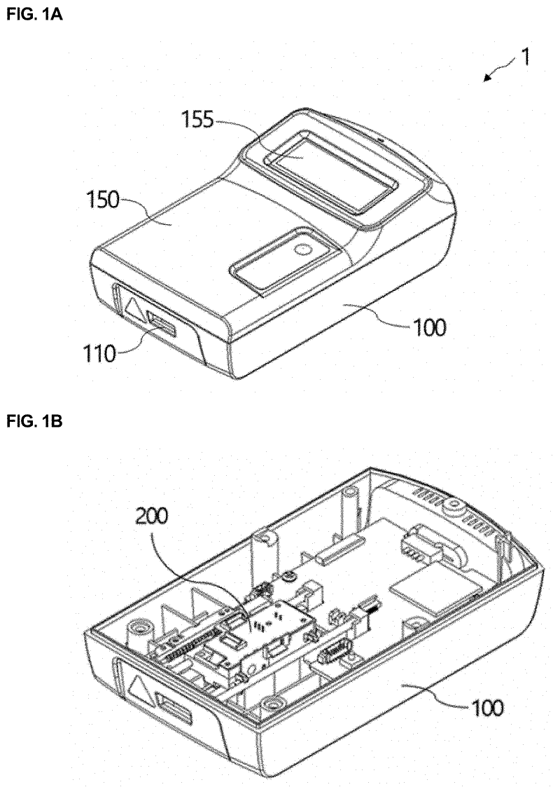

FIGS. 1A and 1B are diagrams illustrating an integrated immunodiagnostic fluorescence reader having multiple diagnosis function1 having a multi-diagnosis function according to the present disclosure. In FIGS. 1A and 1B, an X-axis direction is denoted as a “front and back direction”, a Y-axis direction is denoted as a “lateral direction”, and a Z-axis direction is denoted as a “height direction.” When “the front and back direction”, “the “lateral direction”, or “the height direction” is mentioned, the direction is determined based on a direction indicated in FIGS. 1A and 1B.

FIG. 1A illustrates a state in which an upper frame 150 of the integrated immunodiagnostic fluorescence reader having multiple diagnosis function1 having a multi-diagnosis function is covered. FIG. 1B illustrates a base frame 100 and an internal shape thereof in the state in which the upper frame 150 is not covered.

The integrated immunodiagnostic fluorescence reader having multiple diagnosis function1 having a multi-diagnosis function according to an embodiment of the present disclosure is configured to comprise the base frame 100, the upper frame 150, an optical module 200, a driving module 300, and a reference position detection module 400.

In one embodiment of the present disclosure, the base frame 100 constitutes a lower external appearance of the integrated immunodiagnostic fluorescence reader having multiple diagnosis function1 having a multi-diagnosis function according to the present disclosure. The base frame 100 is configured so that the optical module 200, the driving module 300, the position detection module 400, and various elements are properly fixed or mounted. In one embodiment of the present disclosure, the upper frame 150 constitutes an external appearance of the integrated immunodiagnostic fluorescence reader having multiple diagnosis function1 having a multi-diagnosis function in a form engaged with the base frame 100 to cover the base frame 100, and comprises a display part 155. The display part 155 is preferably integrated and configured with the upper frame 150, but may be configured as a separate frame and combined with the upper frame. In one embodiment of the present disclosure, the display part 155 displays an operating state of the integrated immunodiagnostic fluorescence reader having multiple diagnosis function1 having a multi-diagnosis function according to the present disclosure and results according to an operation thereof through a display window.

In one embodiment of the present disclosure, a given entrance part 110 is formed at one side of the base frame 100. The base frame 100 has a given insertion space 120 opened to the outside through the entrance part 110, and is configured to enable a diagnostic cartridge to be inserted into the insertion space 120 through the entrance part 110.

In one embodiment of the present disclosure, the base frame 100 may comprise a cartridge fixing part 130 as fixing means for preventing a diagnostic cartridge from being unnecessarily shaken or detached when the diagnostic cartridge is inserted into the insertion space 120. Furthermore, the base frame 100 may be provided with a given connector so that a given electric device, a power supply, etc. are connected thereto, but the present disclosure is not limited thereto.

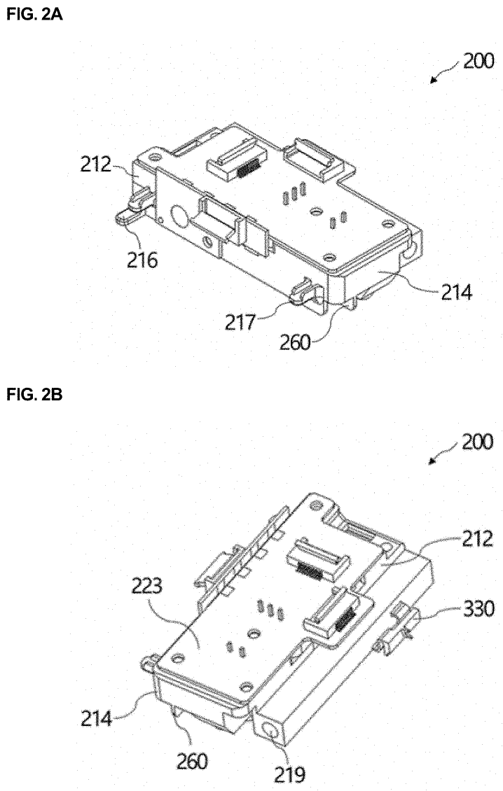

FIGS. 2A and 2B are diagrams illustrating the optical module 200 of the integrated immunodiagnostic fluorescence reader having multiple diagnosis function1 having a multi-diagnosis function according to the present disclosure. FIG. 2A is a diagram viewed from a direction in which one side of the optical module 200, that is, a first guide protrusion 216 and a second guide protrusion 217, are present in the state in which an optical module upper plate 212 has been covered. FIG. 2B is a diagram viewed from a direction in which another aspect of the optical module, that is, a cylinder barrel 219, is present and a driving carrier 330 has been attached.

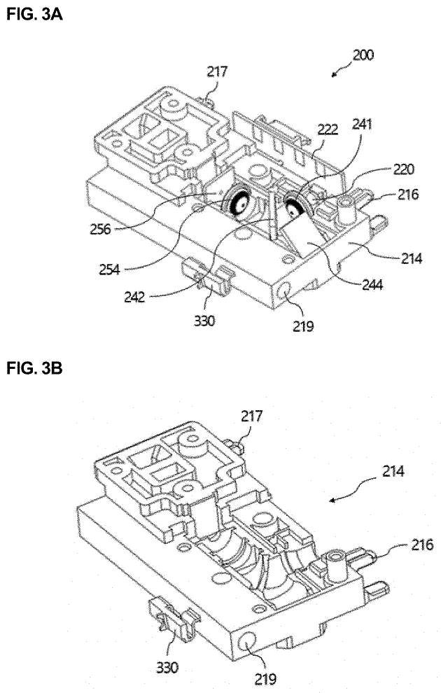

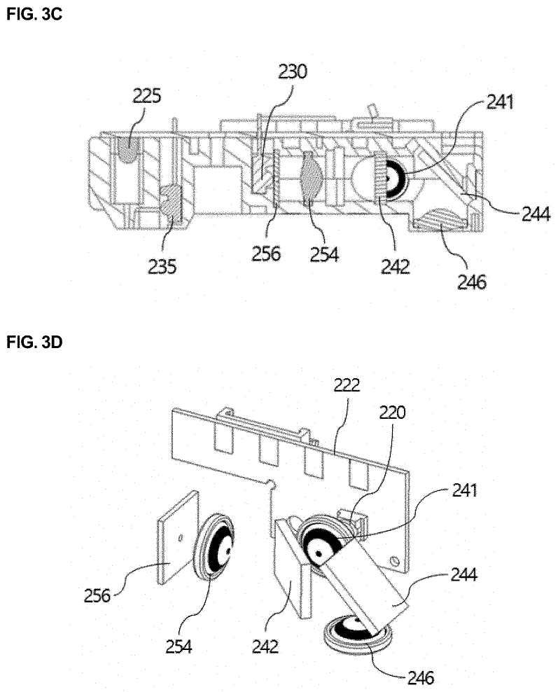

FIGS. 3A-3D are diagrams illustrating parts of the optical module 200 in detail. FIG. 3A is a diagram illustrating an optical module lower part 214, a light source 220, a fluorescence guide part, etc. in the state in which the optical module upper plate 212 has not been covered. FIG. 3B is a diagram illustrating only the optical module lower part 214 in the state in which the light source 220, an ambient light guide part, etc. have not been disposed. FIG. 3C is a cross-sectional view illustrating optical parts, such as the fluorescence guide part, the ambient light guide part, and a light detector 230. FIG. 3D is a perspective view illustrating some of optical parts, such as the light source 220, the fluorescence guide part, etc.

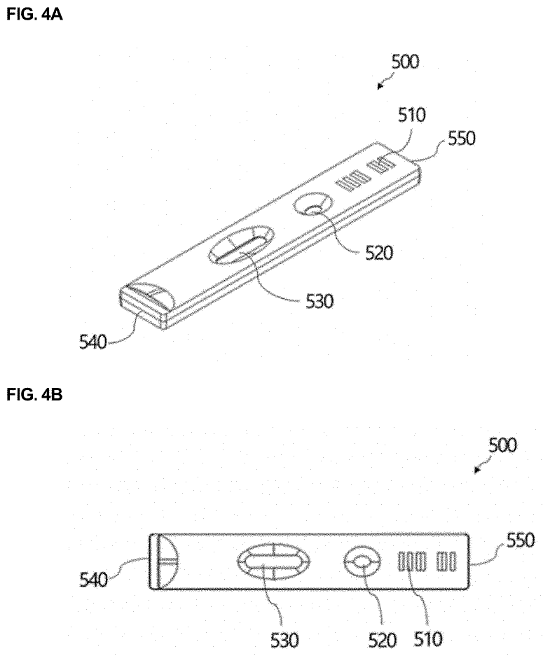

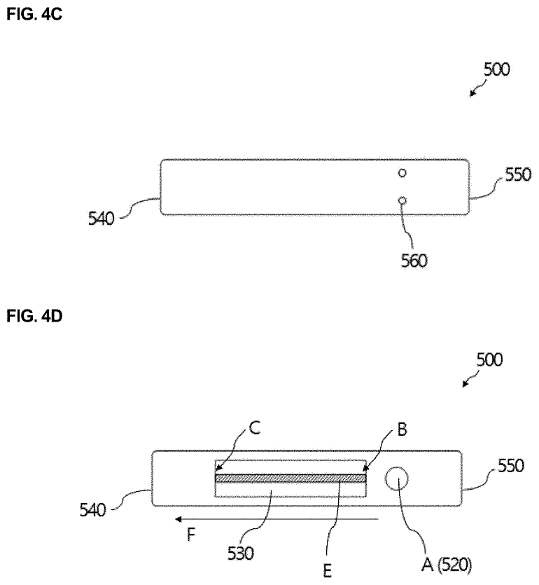

FIGS. 4A-4D are diagrams illustrating a diagnostic cartridge 500 of the integrated immunodiagnostic fluorescence reader having multiple diagnosis function1 having a multi-diagnosis function according to the present disclosure. FIG. 4A is a perspective view of the diagnostic cartridge 500. FIG. 4B is a top plan view of the diagnostic cartridge. FIG. 4C is a plan view of the bottom of the diagnostic cartridge. FIG. 4D is a diagram illustrating a process of a sample being deployed in the diagnostic cartridge.

Referring to FIGS. 4A-4D, the top surface of the diagnostic cartridge 500 comprises the portion of barcode 510 differently assigned for each diagnosis marker and a fluorescence measurement window 530 capable of measuring fluorescence emission light from a fluorescent material changed by an input sample. A sample inlet 520 to which a sample is input is provided between the barcode 510 and the fluorescence measurement window 530. A bottom groove 560 is provided at the bottom of the diagnostic cartridge 500. Among ends of the diagnostic cartridge 500, a side opposite to the barcode 510, that is, a side close to the fluorescence measurement window 530, is called a first end 540, and an end on the side where the barcode 510 is located is called a second end 550.

In one embodiment of the present disclosure, the optical module 200 is provided to radiate a given light to the diagnostic cartridge 500 inserted into the base frame 100 and to detect fluorescence emission light and barcode-ambient light of the incident light emitted after the incident light is radiated to the fluorescence measurement window 530 and barcode 510 of a sample.

In one embodiment of the present disclosure, the barcode reading system may use commercial one-dimensional or two-dimensional barcode. However, the one-dimensional barcode has a disadvantage in that a price rises due to increased complexity of a device in reading a close cartridge because a corresponding structure is large and a focal distance is long. The two-dimensional barcode may be properly selected with complexity because it comprises a large amount of information compared to the one-dimensional barcode, and requires a high-performance processor and an image processing technique for two-dimensional decoding. For this reason, a cost may rise because a CMOS or CCD camera module is used. Accordingly, in one embodiment of the present disclosure, multiple diagnosis markers can be identified using unique one-dimensional barcode specialized for cartridge identification without using commercial barcode. To this end, a method of identifying a difference between signals of the unique one-dimensional barcode using a light source and a detection unit and classifying the signals in a binary number unit may be used. In one embodiment of the present disclosure, the unique one-dimensional barcode may have a shape in which a plurality of solid-line parts are isolated at predetermined intervals, and may preferably comprise five or more solid-line parts, but the present disclosure is not limited thereto.

In one embodiment of the present disclosure, the optical module 200 may be configured to comprise the module upper plate 212 and the module lower part 214 forming a module casing, the light sources 220 and 225, the fluorescence guide part, the ambient light guide part, the light detectors 230 and 235, an interrupt protrusion part 260, and various PCBs and connectors.

The module casing is configured as a casing having a given box form, has a space formed therein, and is configured to have the light source 220, the fluorescence guide part, the ambient light guide part, and the detectors 230 and 235 embedded and installed therein. In one embodiment of the present disclosure, the module casing has a stereoscopic shape having a generally rectangular box form, and preferably has a construction coupled by the module lower part 214 and the module upper plate 212 that covers and fixes an opened top of the module lower part 214, but the present disclosure is not essentially limited thereto.

Various boards and connectors may be provided on the outside of the module casing. In one embodiment of the present disclosure, the connectors are provided so that the light sources 220 and 225, the detectors 230 and 235, etc. within the module casing exchange electrical signals with an external electrical device, and may be connected to given electrical terminals.

The module casing, in particular, the module lower part 214 may be equipped with given guide parts disposed on the outside of a side thereof on both sides. In one embodiment of the present disclosure, the guide part is a member for stably guiding a displacement of the module casing when the module casing is displaced in the front and back direction by the driving module 300 to be described later.

The guide part comprises a cylinder barrel 219 on one side thereof and guide protrusions 216 and 217 on the other side thereof so that two guide shafts 352 and 354 to be described later are guided. That is, the cylinder barrel 219 that guides the first guide shaft 352 to be inserted in a lengthwise direction thereof is provided on one side of the module lower part 214. The first guide protrusion 216 and the second guide protrusion 217 guided along the second guide shaft 354 are provided on the other side of the module lower part 214. However, the first guide shaft 352 does not need to be essentially guided only in the form of a cylinder barrel, and may be guided in the form of a guide protrusion like the second guide shaft 354.

In one embodiment of the present disclosure, the first guide protrusion 216 is preferably configured to comprise two upper and lower protrusions at one end on one side where the second guide shaft 354 is located. The second guide protrusion 217 is preferably configured as one protrusion at the other end on one side where the second guide shaft 354 is located, but the present disclosure is not essentially limited thereto. An upper and down width between the upper protrusion and lower protrusion of the first guide protrusion 216 may correspond to the diameter of the second guide shaft 354.

In one embodiment of the present disclosure, the light source 220 and/or the barcode light source 225 are disposed within the module lower part 214 of the module casing, and may generate a light having a given wavelength by being applied with given power. For example, the light source 220 and the barcode light source 225 may be configured as light-emitting diodes (LED) for generating light having a specific wavelength band or may be configured as given laser light sources for generating straight-line light, but the present disclosure is not essentially limited thereto.

In an embodiment of the present disclosure illustrated in the drawings, a case where the light source 220 and the barcode light source 225 are separately provided, that is, two light sources are present, is described as an example, but the present disclosure is not essentially limited thereto. That is, the present disclosure may be configured so that the light source comprises the one light source 220 to also play a role as the barcode light source. In one embodiment of the present disclosure, if the two light sources are used, the light source board 222 for fluorescence for controlling an operation of the light source 220 used as a light source for fluorescence and the light source board 223 for barcode for controlling an operation of the barcode light source 225 may be provided.

In one embodiment of the present disclosure, the fluorescence guide part is disposed within the module casing to guide a light generated by the light source 220 to the fluorescence measurement window 530 of the diagnostic cartridge 500, and to guide fluorescence emitted from the fluorescence measurement window to the fluorescence detector 230. Accordingly, the light generated by the light source 220 is guided by the fluorescence guide part, radiated to the fluorescence measurement window 530 of the diagnostic cartridge 500, and incident on a given fluorescent material comprised in the sample loaded onto the diagnostic cartridge 500, so that fluorescence emission light is generated by the fluorescent material.

The ambient light guide part is disposed within the module casing to guide a light generated by the barcode light source 225 to the barcode 510 of the diagnostic cartridge 500, and to guide ambient light, such as reflected light, diffused light, and scattered light reflected from the barcode 510, to the ambient light detector 235. In an embodiment of the present disclosure illustrated in the drawings, the ambient light guide part has been illustrated as being comprised separately from the fluorescence guide part. However, if the number of light sources is one, the fluorescence guide part may be configured to partially play a role as the ambient light guide part.

The fluorescence detector 230 and the ambient light detector 235 are configured to obtain fluorescence emission light and ambient light generated from the diagnostic cartridge 500. In one embodiment, photodiodes are used as the fluorescence detector 230 and the ambient light detector 235. Fluorescence emission light and ambient reflected light are converted into an electrical signal through the photodiodes. In another embodiment, the fluorescence detector 230 and the ambient light detector 235 may be composed of different types of photo detectors.

In an embodiment of the present disclosure illustrated in the drawings, a case where the fluorescence detector 230 and the ambient light detector 235 are separately provided, that is, two light detectors are present, is described as an example, but the present disclosure is not essentially limited thereto. That is, the present disclosure may be configured so that the fluorescence detector comprises only the one light detector 230 to also play a role as the ambient light detector.

The interrupt protrusion part 260 may be provided to protrude at one end at the bottom of the module lower part 214. The interrupt protrusion part 260 is configured as a protrusion part that protrudes with a given width and length. The interrupt protrusion part 260 controls an operation of an interrupt sensor part 420 to be described later, and a detailed configuration and operation of the interrupt sensor part 420 are described later.

Hereinafter, if the optical module 200 according to an embodiment of the present disclosure comprises the two light sources 220 and 225 and the two light detectors 230 and 235 as illustrated in the drawings, the fluorescence guide part and the ambient light guide part are described in detail.

In one embodiment of the present disclosure, the fluorescence guide part comprises a lens for light source 241, a first mirror 242, a second mirror 244, a downward lens 246, a detection lens 254, and a pin hole 256. The lens for light source 241 concentrates irradiation light emitted in a horizontal and lateral direction from the light source 220, and sends the light to the first mirror 242. The first mirror 242 changes the path of the irradiation light concentrated by the lens for light source 241 so that the irradiation light travels in a horizontal and front direction, and reflects the light. That is, the first mirror 242 is inclined laterally to the path of light laterally emitted by the light source 220 so that the path of the light is changed to the front direction. Specifically, assuming that light radiated by the light source 220 is incident on an XY plane in a Y-axis direction, the first mirror 242 may have an orientation angle that is perpendicular to the XY plane and inclined by 45° with respect to the Y-axis. The first mirror 242 is a dichroic mirror and has both a filter function and a reflection mirror function. That is, the first mirror 242 is an element that reflects a wavelength band of irradiation light and transmits a wavelength band of fluorescence emission light.

The second mirror 244 changes the path of the light whose path has been changed by the dichroic first mirror 242 in a perpendicular and downward direction, and sends the light toward the downward lens 246 and the fluorescence measurement window 530 of the diagnostic cartridge 500. The second mirror 244 is a total reflection mirror, and is downward inclined to downward change the path of light radiated in the front direction. Specifically, assuming that light reflected by the first mirror 242 is incident on the XY plane in the X-axis direction, the second mirror 244 may have an orientation angle that is perpendicular to the XZ plane and inclined by 45° with respect to the X axis.

The downward lens 246 concentrates the light whose path has been changed by the second mirror 244, and radiates the concentrated light to the fluorescence measurement window 530 of the diagnostic cartridge 500. Furthermore, the downward lens 246 concentrates fluorescence emission light generated due to the light radiated to the fluorescence measurement window 530, and sends the concentrated light to the second mirror 244 again. The fluorescence emission light concentrated by downward lens 246 is reflected by the second mirror 244, so that the fluorescence emission light having a direction changed is sent to the first mirror 242 again. The first mirror is a dichroic mirror, and thus directs the fluorescence emission light toward the detection lens 254 by transmitting the fluorescence emission light without reflecting the fluorescence emission light. That is, the fluorescence emission light transmits straight through the first mirror 242. The detection lens 254 concentrates the fluorescence emission light passing through the first mirror and directs the concentrated light toward the pin hole 256.

In one embodiment of the present disclosure, the pin hole 256 filters, transmits, and guides the fluorescence emission light concentrated by the detection lens 254. The fluorescence emission light filtered by the pin hole 256 is received by the fluorescence detector 230. The pin hole 256 may be configured as a given hole that enables only fluorescence emission light within a specific path to be incident on the fluorescence detector 230. Each of the lenses 241, 246, and 254 may be configured as a given device that properly modulates and converts light or fluorescence emission light. The fluorescence detector 230 is equipped with a converter. Accordingly, light received by the fluorescence detector 230 is converted into a voltage signal through the converter.

In the configuration of the embodiment comprising the two light sources 220 and 225 and the two light detectors 230 and 235, the ambient light guide part does not comprise a separate lens, a separate mirror, etc. That is, the ambient light guide part directly guides, to the barcode 510 of the diagnostic cartridge 500, light generated by the barcode light source 225, and guides ambient light reflected from the barcode 510 to be perpendicularly incident on the ambient light detector 235.

Although not illustrated in the drawing in one embodiment of the present disclosure, in another embodiment of the present disclosure comprising only the one light source 220, the ambient light guide part is also differently configured. That is, the ambient light guide part shares the path of light incident on the barcode with the fluorescence guide part, so that light radiated by the light source 220 is incident on the barcode 510 of the diagnostic cartridge 500 via the lens for light source 241, the first mirror 242, the second mirror 244, and the downward lens 246. However, ambient light reflected from the barcode 510 does not pass through the first mirror 242, that is, the dichroic mirror of the fluorescence guide part. For this reason, a separate lower pin hole (not illustrated) is provided to adjust the path of ambient light reflected from the barcode so that the light is perpendicularly incident on the light detector 230.

In this case, that is, if one light source is present, one light detector may be configured to perform both fluorescence detection and ambient light detection functions. Two detectors, that is, the fluorescence detector 230 and the ambient light detector 235, may be configured. Regardless of the number of detectors, a light detector that receives light around the barcode may comprise a lower pin hole (not illustrated) thereunder, and may filter, transmit, and guide the received light. Accordingly, if only one light source 220 is provided, the ambient light guide part comprises optical elements such as the lower pin hole.

Although not illustrated, in one embodiment of the present disclosure, another embodiment of the present disclosure comprising the two light sources 220 and 225 and one light detector 230 is possible. In this case, the ambient light guide part is differently configured. If two light sources, that is, the light source 220 for fluorescence radiation and the separate barcode light source 225 are provided, the ambient light guide part does not need to share the path of light incident on the barcode with the fluorescence guide part. Accordingly, similar to the case comprising the two light sources 220 and 225 and the two light detectors 230 and 235, light generated by the barcode light source 225 is directly guided to the barcode 510 of the diagnostic cartridge 500, and ambient light reflected from the barcode 510 is guided to be perpendicularly incident on the fluorescence detector 230.

However, since the ambient light detector is configured to also perform the fluorescence detection function, the lower pin hole needs to be provided to filter, transmit, and guide ambient light. For example, if the two light sources 220 and 225 and the one light detector 230 are provided, the ambient light guide part needs to comprise the lower pin hole.

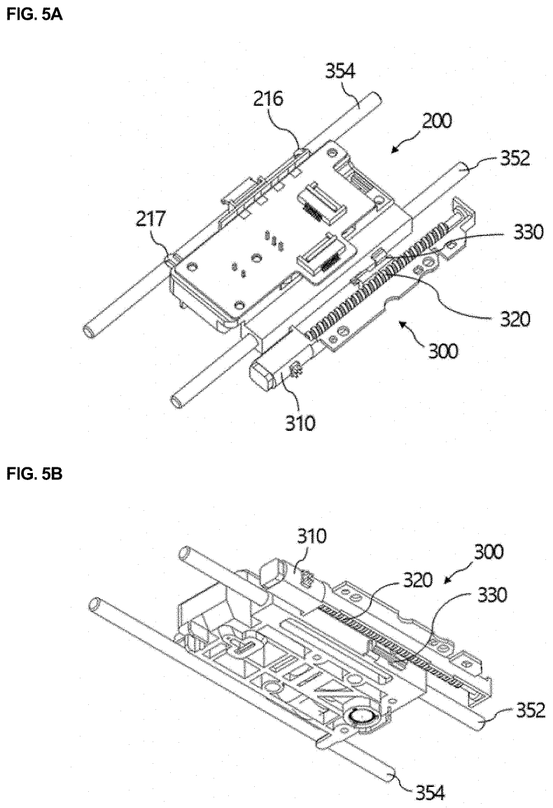

FIGS. 5A and 5B are diagrams illustrating the optical module 200 and driving module 300 of the integrated immunodiagnostic fluorescence reader having multiple diagnosis function1 having a multi-diagnosis function according to the present disclosure. FIG. 5A is a perspective view of a structure in which the optical module 200 and the driving module 300 are coupled, which is viewed from the top. FIG. 5B is a perspective view of the structure in which the optical module 200 and the driving module 300 are coupled, which is viewed from the bottom.

In one embodiment of the present disclosure, the driving module 300 consists of a device that provides or delivers motive power to displace the optical module 200 or a device that guides a displacement of the optical module 200. Specifically, the driving module 300 may be configured to comprise a motor 310, a driving shaft 320, a driving carrier 330, and guide shafts 352 and 354.

In one embodiment of the present disclosure, the motor 310 is provided to generate a rotatory power by receiving given electric power. The motor 310 may be disposed at an edge portion on one side within the internal space of the casing.

The driving shaft 320 is configured as a given shaft that is coupled to the motor 310 and rotated. The driving shaft 320 is disposed to elongate front and back in parallel to the optical module 200 on at least one outside of the optical module 200. In one embodiment of the present disclosure, the driving carrier 330 is a member coupled to one side of the optical module 200, and is a member that couples the optical module 200 and the driving shaft 320 and that achieves a front and back displacement of the driving module 300 using a rotatory power delivered to the driving shaft 320. One side of the driving carrier 330 is connected to the optical module 200. A given elastic part (no reference numeral) may be provided between one side and the other side of the driving carrier 330.

In one embodiment of the present disclosure, one side of the driving carrier 330 is configured to be fixed to the module lower part 214 of the optical module casing, and is preferably fixed to one side where the cylinder barrel 219 is located among sides of the module lower part 214. Furthermore, the driving carrier 330 and the optical module 200 may be coupled and integrated. When an external force is applied to the driving carrier 330, the carrier 330 and the optical module 200 may be displaced at the same time.

The guide shafts 352 and 354 may be configured as given beams that are disposed on both sides on the outside of the optical module 200, respectively, and that are elongated front and back. For example, the first guide shaft 352 located between the driving shaft 320 and the optical module 200 and the second guide shaft 354 located on a side opposite to the side where the first guide shaft 352 is located may be provided. The two guide shafts 352 and 354 each have one end fixed to the guide shaft fitting groove 145 of the base frame 100 to be described later and the other end cradled in the guide shaft mounting groove 140 of the base frame, so the two guide shafts are disposed in parallel to the lengthwise direction of the diagnostic cartridge 500 inserted into the insertion space 120.

As described above, the guide parts are provided on both sides of the module lower part 214 of the casing of the optical module 200. The cylinder barrel 219 is provided on one side of the optical module 200, that is, the side to which the driving carrier 330 is coupled and fixed, enabling the first guide shaft 352 to be inserted and guided in the lengthwise direction thereof. The first guide protrusion 216 and the second guide protrusion 217 are provided on the other side of the optical module 200 and guided along the second guide shaft 354.

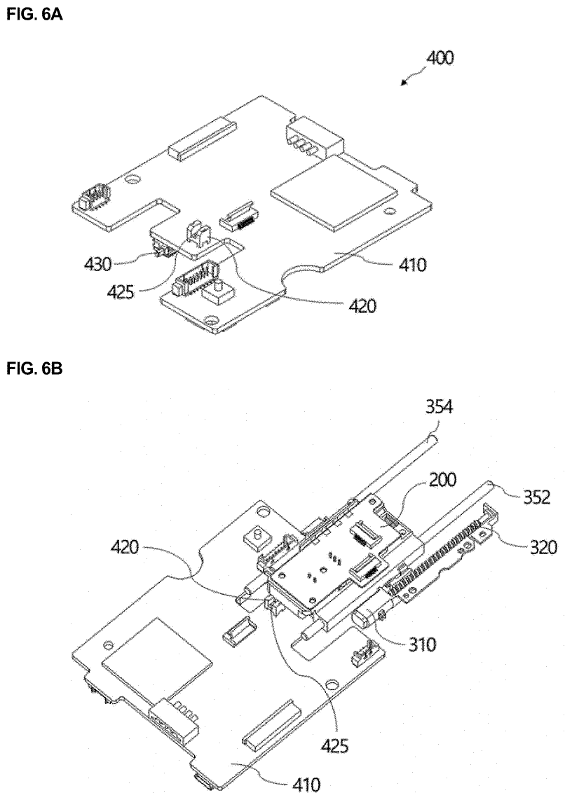

FIGS. 6A and 6B are diagrams illustrating the reference position detection module of the integrated immunodiagnostic fluorescence reader having multiple diagnosis function1 having a multi-diagnosis function according to the present disclosure. FIG. 6A is a perspective view illustrating only the reference position detection module. FIG. 6B is a perspective view illustrates a shape in which the reference position detection module 400 and the optical module 200 are connected.

The reference position detection module 400 is configured to comprise a sensor that detects the insertion of the diagnostic cartridge 500 and a position of the optical module 200. The reference position detection module 400 is configured to comprise a module main board 410, an interrupt sensor part 420, and a cartridge sensing switch 430.

The module main board 410 may be configured as a given PCB that is located within the base frame 100 and fixedly coupled thereto. Preferably, the module main board 410 may be disposed at a position in a direction opposite to the entrance part 110 on the side at the front of the base frame 100. The interrupt sensor part 420 may be disposed over the module main board 410, and the cartridge sensing switch 430 may be disposed under the module main board 410. Furthermore, a given connector may be provided.

In one embodiment of the present disclosure, the interrupt sensor part 420 has a given insertion groove formed at the front thereof, and is composed of an insertion detection sensor 425 located within the groove. That is, the interrupt sensor part 420 has the insertion groove formed at the front thereof, and part of the front is depressed. The interrupt sensor part 420 may be composed of a given insertion detection sensor 425 from which a signal is generated or by which a signal is blocked when a given device or obstacle is inserted into the insertion groove.

The interrupt protrusion part 260 provided in the optical module 200 may be inserted into the insertion groove formed in the interrupt sensor part 420. A distance between the interrupt sensor part 420 and the interrupt protrusion part 260 varies as the optical module 200 is displaced. When the optical module 200 becomes adjacent to the module main board 410, the interrupt protrusion part 260 is inserted into the interrupt sensor part 420. The insertion detection sensor 425 of the interrupt sensor part 420 may generate a given signal or stop a signal when the interrupt protrusion part 260 is inserted into the interrupt sensor part 420. Specifically, the interrupt sensor part 420 according to the present application may detect the out-of phase of the optical module 200. The out-of phase means that the optical module 200 is not accurately moved along a rail by an instructed step. In order to solve the out-of phase, a position that becomes a reference is determined, and the optical module 200 is moved from the position by an instructed step. The interrupt sensor part 420 enables the reference position to be recognized. That is, when the interrupt protrusion part 260 provided in the optical module 200 is inserted into the insertion groove formed in the interrupt sensor part 420, the reference position is recognized. The optical module first moves to the reference position before moving from one point to another point, and then moves from the reference position by an instructed step.

In one embodiment of the present disclosure, the cartridge sensing switch 430 is configured as a given switch that senses whether the diagnostic cartridge 500 has been inserted into an accurate depth within the base frame 100. For example, when the diagnostic cartridge 500 is inserted up to a given depth and reaches a position where test can be properly performed, the sensing switch 430 may be pressed by the diagnostic cartridge 500 to generate a signal, which is described in detail later.

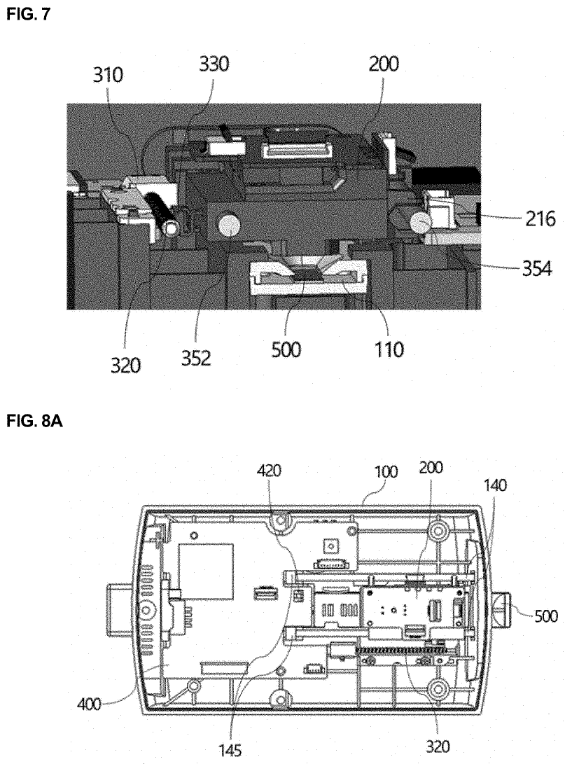

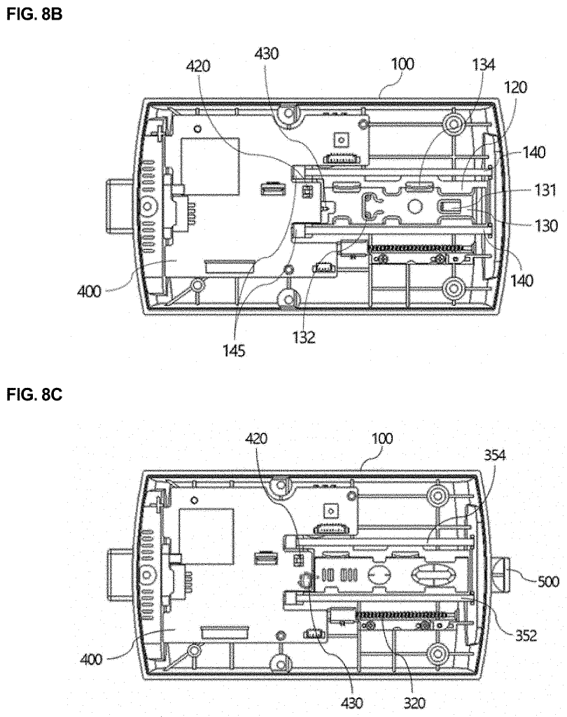

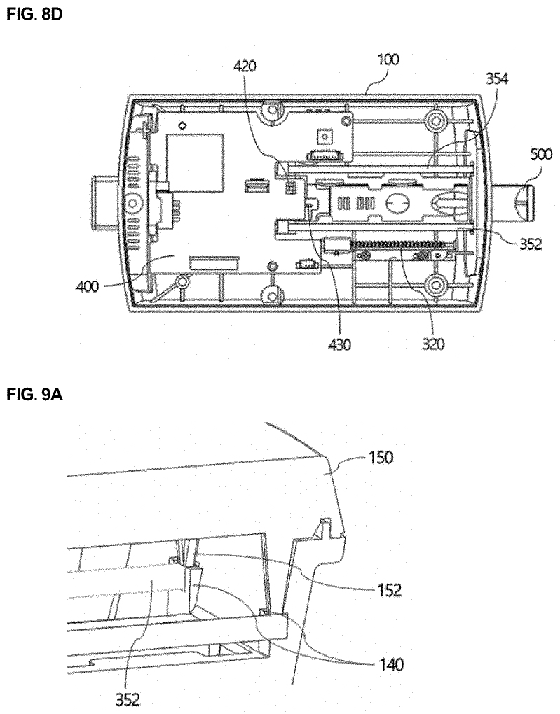

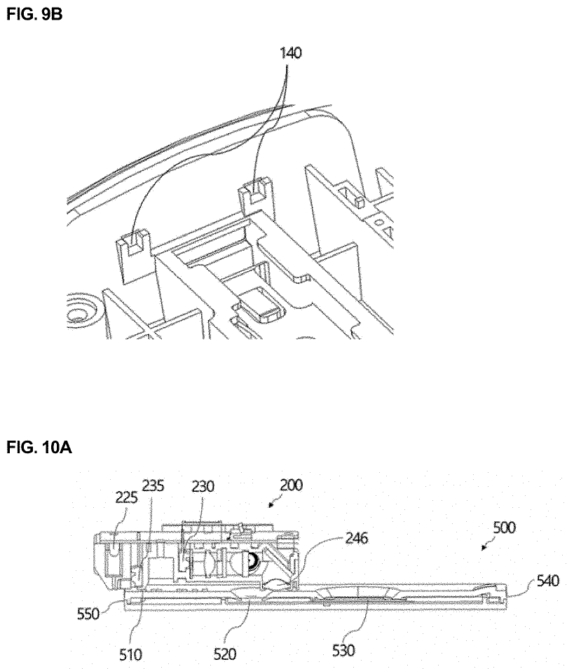

FIG. 7 is a diagram illustrating one cross section of the integrated immunodiagnostic fluorescence reader having multiple diagnosis function1 having a multi-diagnosis function according to the present disclosure, on which the optical module 200 and the driving module 300 are mounted. FIGS. 8A-8D are plan views illustrating the base frame 100 of the integrated immunodiagnostic fluorescence reader having multiple diagnosis function1 having a multi-diagnosis function according to the present disclosure. FIG. 8A is a plan view of a state in which the optical module 200 is mounted on and the diagnostic cartridge 500 is inserted into the base frame 100. FIG. 8B is a plan view of a state in which the optical module 200 and the diagnostic cartridge 500 are not mounted on the base frame 100. FIG. 8C is a plan view illustrating a state in which the diagnostic cartridge 500 has been inserted to the end of the base frame in the state in which the optical module has not been mounted on the base frame. FIG. 8D is a plan view illustrating a state right before the diagnostic cartridge 500 is inserted to the end of the base frame. FIGS. 9A and 9B are diagrams illustrating a structure in which the guide shafts 352 and 354 are fixed by the base frame 100 and the upper frame 150.

In one embodiment of the present disclosure, the base frame 100 has an opened top surface, and comprises the entrance part 110 into which the diagnostic cartridge 500 is inserted on a side at the front thereof and the insertion space 120 opened to the outside through the entrance part 110 on the side at the front. Furthermore, a space for the movement of the optical module is located on the insertion space 120, so that the optical module is moved along the two guide shafts 352 and 354 fixed to both sides in parallel to the lengthwise direction of the diagnostic cartridge 500 inserted into the insertion space 120.

In one embodiment of the present disclosure, a space for driving module mounting is located on one side of the space for the movement of the optical module. A space for reference position detection module mounting that detects the reference position of the optical module 200 is also provided within the base frame 100.

The upper frame 150 covers the opened top of the base frame 100 and fixes the top thereto. The guide shaft fixing protrusion 152 fixes the end of each of the guide shafts 352 and 354 along with the guide shaft mounting groove 140 of the base frame 100. That is, one end of each of the guide shafts 352 and 354 is fit into and fixed by the guide shaft fitting groove 145 of the base frame. The other ends of the guide shafts 352 and 354 are cradled in the guide shaft mounting groove 140 of the base frame 100 and simultaneously pressed and fixed by the guide shaft fixing protrusion 152. The upper frame 150 comprises a fastening part fastened to the base frame 100 in order to maintain the fixing of the guide shaft fixing protrusion 152.

The cartridge fixing part 130 is provided in the insertion space 120 under the space for the movement of the optical module within the base frame 100. When the diagnostic cartridge 500 is inserted into the insertion space 120, the cartridge fixing part 130 fixes the position of the diagnostic cartridge 500 by applying pressure to at least part of the diagnostic cartridge.

Referring to FIG. 8B, the cartridge fixing part 130 of the base frame 100 comprises a fixing spring 131, a lifting-up protrusion 132, and a side fitting frame 134. The fixing spring 131 is a member having an elastic force, and lifts up and fixes the bottom of the diagnostic cartridge 500. The lifting-up protrusion 132 is matched with a portion of the bottom groove 560 of the diagnostic cartridge 500, and helps the fixing. The side of the diagnostic cartridge 500 is inserted into the side fitting frame 134, and thus the diagnostic cartridge 500 is fixed by the side fitting frame 134.

Referring to FIGS. 8C and 8D, when the diagnostic cartridge 500 is inserted up to a proper depth within the insertion space 120 through the entrance part 110 on the side at the front thereof and reaches a regular position, the second end 550 of the diagnostic cartridge 500 generates an insertion signal for the diagnostic cartridge 500 while pressing the cartridge sensing switch 430 by pushing the cartridge sensing switch 430. In this case, the cartridge sensing switch 430 may be configured to comprise a button having a given shape so that the cartridge sensing switch 430 has a construction to be pushed and pressed by the second end 550 of the diagnostic cartridge 500.

As described above, the interrupt sensor part 420 is provided over the module main board 410, the cartridge sensing switch 430 is provided under the module main board 410, and the interrupt sensor part 420 and the cartridge sensing switch 430 are integrated with the module main board 410. Accordingly, a reduction in position accuracy attributable to an assembly error can be prevented. That is, for example, if the interrupt sensor part 420 and the cartridge sensing switch 430 are independently moved or independently assembled, an error occurrence possibility may rise. In contrast, according to the present disclosure, an error occurrence possibility can be reduced and accurate test can be performed because the interrupt sensor part 420 detecting a position of the optical module 200 and the cartridge sensing switch 430 detecting a position of the diagnostic cartridge 500 are together provided in the one module main board 410.

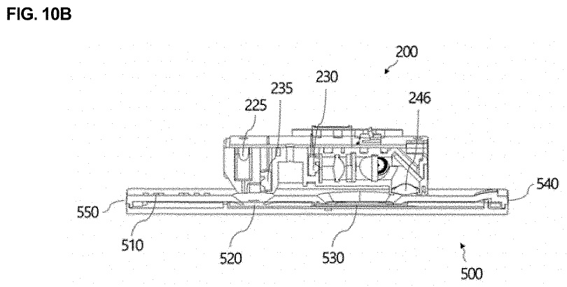

Hereinafter, an operation of the integrated immunodiagnostic fluorescence reader having multiple diagnosis function1 having a multi-diagnosis function according to the present disclosure is described. FIGS. 10A and 10B are diagrams illustrating part of a cross section of the integrated fluorescence reader for multi-diagnosis on which the diagnostic cartridge 500 is mounted. FIG. 10A is a cross-sectional view if the optical module 200 reads the barcode 510 of the diagnostic cartridge 500. FIG. 10B is a cross-sectional view if the optical module 200 reads the fluorescence measurement window 530 of the diagnostic cartridge 500.

When the diagnostic cartridge 500 is inserted, first, the optical module 200 moves to a position where the barcode 510 of the diagnostic cartridge 500 can be properly read. That is, at a position where light emitted from the barcode light source 225 can be properly radiated to the barcode 510 of the diagnostic cartridge 500 through the ambient light guide part, the barcode detector 235 may receive light reflected from the barcode 510 and identify a diagnosis marker, the type of sample, etc. based on assigned barcode information.

After the barcode 510 of the diagnostic cartridge 500 is read, the optical module 200 moves to a position where the fluorescence measurement window 530 of the diagnostic cartridge 500 can be properly read. That is, the position is a location where light emitted by the light source 220 of the optical module can be properly radiated to the fluorescence measurement window 530 of the diagnostic cartridge 500 through the fluorescence guide part. The light is finally radiated to the sample in the portion of the fluorescence measurement window 530 of the diagnostic cartridge 500 located under the fluorescence measurement window 530 through the downward lens 246.

The sample of the diagnostic cartridge 500 comprises a given fluorescent material and thus generates fluorescence emission light when the light is incident on the sample. The fluorescence emission light is upward emitted. As described above, the fluorescence emission light passes through the downward lens 246 and is received by the fluorescence detector 230 via the second mirror 244, the first mirror 242, the detection lens 254, and the pin hole 256.

As illustrated in FIG. 4D, after lateral flow start time of the sample is detected at the start point B of the fluorescence measurement window 530, the sample moves to the end point C of the fluorescence measurement window 530. The speed of the lateral flow is calculated by detecting lateral flow end time of the sample at the end point C of the fluorescence measurement window 530.

FIGS. 10A and 10B shows an example in the case where the two light sources 220 and 225 and the two light detectors 230 and 235 are provided in an embodiment of the present disclosure. However, a similar operation is performed only when one light source or one light detector is present. In this case, a movement distance of the optical module 200 may be further increased.

Hereinafter, a displacement of the optical module 200 by the driving module 300 is described.

As described above, the driving carrier 330 is fixed to the module lower part 214 of the optical module 200. The driving carrier 330 and the driving shaft 320 are connected. When the driving shaft 320 is rotated by a rotatory power of the motor 310, the driving carrier 330 is displaced in the front and back direction. The optical module 200 coupled to the driving carrier 330 may be displaced in the front and back direction.

Due to such a configuration, a displacement distance between the carrier 330 and the optical module 200 can be controlled by the number of rotations of the motor 310, so that accurate control over a displacement distance of the optical module 200 can be performed. Furthermore, as described above, motive power can be effectively delivered because the carrier 330 can be strongly attached to the driving shaft 320, and accurate control over the displacement distance can be more effectively achieved. Moreover, the detachment, out-of phase, etc. of the optical module 200 can be prevented because the guide shafts 352 and 354 are further provided to stably guide a displacement of the optical module 200.

The immunodiagnostic fluorescence reader1 according to the present application comprises an optimal construction for an implementation of the aforementioned method, and may be implemented as hardware, firmware, or software for the implementation of the method or a combination of them. If the method is implemented as software, a storage medium comprises a given medium for storage or delivery in a form readable by a device, such as a computer. For example, the computer-readable medium comprises a read only memory (ROM); a random access memory (RAM); a magnetic disk storage medium; an optical storage medium; a flash memory device, other electrical, optical, or acoustic signal delivery media, etc.

Furthermore, although the preferred embodiments have been illustrated and described above, this specification is not limited to the aforementioned specific embodiments, and a person having ordinary skill in the art to which this specification pertains may modify the present disclosure in various ways without departing from the gist of the present disclosure in the claims. Such modified embodiments should not be individually understood from the technical spirit or prospect of this specification.

[Description of reference Numerals]

1: fluorescence reader for immunodiagnosis
100: base frame
110: entrance part on side at front
120: inner space
130: cartridge fixing part
131: fixing spring
132: lifting-up protrusion
134: side fitting frame
140: guide shaft mounting groove
145: guide shaft fitting groove
150: upper frame
152: guide shaft fixing protrusion
155: display part
200: optical module
212: module upper plate
214: module lower plate
216: first guide protrusion
217: second guide protrusion
219: cylinder barrel
220: light source
222: light source board for fluorescence
223: light source board for barcode
225: barcode light source
230: fluorescence detector
235: ambient light detector
241: lens for light source
242: first mirror
244: second mirror
246: downward lens
254: detection lens
256: pin hole
260: interrupt protrusion part
300: driving module
310: motor
320: driving shaft
330: driving carrier
352: first guide shaft
354: second guide shaft
400: reference position detection module
410: module main board
420: interrupt sensor part
425: inserted sensor
430: cartridge sensing switch
500: diagnostic cartridge
510: barcode
520: sample inlet
530: fluorescence measurement window
540: first end
550: second end
560: bottom groove

1. An integrated immunodiagnostic fluorescence reader comprising:
a base frame with an open top surface, comprising: an entrance part at a front side thereof into which a diagnostic cartridge having a barcode differently assigned to each diagnosis marker and a fluorescence measurement window is inserted wherein the barcode is located on the same side as the fluorescence measurement window; an insertion space opened to an outside through the entrance part, wherein the cartridge inserted through the entrance part; a space for the movement of an optical module, being an upper part of the inner space in which an optical module moves along two guide shafts each fixed to the base frame in parallel to a lengthwise direction of the diagnostic cartridge located in the inner space; a space for driving module mounting, being located on one side of the space for the movement of the optical module; a space for reference position detection module mounting wherein the detection module detects a reference position of the optical module; a cartridge fixing part fixing the diagnostic cartridge at a position by applying pressure at least part of the diagnostic cartridge when the diagnostic cartridge is inserted into the inner space; fitting grooves into each of which one end of each of the two guide shafts is fit and fixed and mounting grooves into each of which the other end of each of the two guide shafts is mounted;
an optical module located in the space for the movement of an optical module of the base frame and configured to radiate light to the fluorescence measurement window and barcode of the diagnostic cartridge while moving in parallel along the two guide shafts and to detect fluorescence light and ambient light reflected by the radiated light;
a driving module located in the space for driving module mounting of the base frame and connected to the optical module to move the optical module in parallel;
a detection module for a reference position located in the space for reference position detection module mounting of the base frame and configured to detect a reference position of the optical module and to detect whether the cartridge is inserted; and
an upper frame configured to cover and fix the opened top surface of the base frame comprising a guide shaft fixing protrusion fixing one end of each of the guide shafts together with the guide shaft mounting groove of the base frame and a fastening part fastened to the base frame in order to maintain the fixing by the guide shaft fixing protrusion.

2. The integrated immunodiagnostic fluorescence reader of claim 1, wherein the optical module comprises:
a module casing comprising a module lower part and a module upper plate covering and fixing an opened top surface of the module lower part to form an box shaped internal space;
a light source disposed within the module casing;
an ambient light guide part disposed within the module casing and configured to guide a light from the light source to the barcode of the diagnostic cartridge and to guide an ambient light reflected from the barcode to a light detector;
a fluorescence guide part disposed within the module casing comprising a dichroic mirror for guiding a light from the light source to the fluorescence measurement window of the diagnostic cartridge and for guiding a fluorescence emitted from the fluorescence measurement window to a light detector; and
a light detector configured to detect the ambient light and the fluorescence;
wherein the module lower part on one side thereof has a cylinder barrel into which one of the two guide shafts is put in to guide the optical module in a lengthwise direction, and on the other side thereof has guide protrusions to guide the optical module along the other of the two guide shafts, and on the bottom side thereof has an interrupt protrusion part being inserted into the detection module for a reference position to determine the reference position.

3. The integrated immunodiagnostic fluorescence reader of claim 2, wherein the fluorescence guide part comprises:
a lens for light source for concentrating irradiation light emitted from the light source in a horizontal lateral direction;
a dichroic first mirror for changing a path of the irradiation light concentrated by the lens for light source so that the irradiation light travels in a horizontal and front direction, and for transmitting straight the fluorescence emitted from the fluorescence measurement window of the diagnostic cartridge;
a second mirror for changing a path of the light whose path has been changed by the dichroic first mirror in a vertical and downward direction to send the light to the fluorescence measurement window of the diagnostic cartridge, and for sending the fluorescence to the first mirror, generated from the fluorescence measurement window of the diagnostic cartridge;
a downward lens for concentrating the light whose path has been changed by the second mirror, irradiating the concentrated light to the fluorescence measurement window of the diagnostic cartridge, concentrating the fluorescence emitted from the fluorescence measurement window, and sending the concentrated fluorescence to the second mirror;
a detection lens for concentrating the fluorescence whose path has been changed by the second mirror and transmitted straight the first mirror, and concentrated by the downward lens; and
a pin hole for filtering, transmitting and guiding the fluorescence concentrated by the detection lens.

4. The integrated immunodiagnostic fluorescence reader of claim 3,
wherein the light source is one,
wherein the ambient light guide part shares a path of light incident on the barcode with the path of the fluorescence guide part, and further comprises a mirror for adjusting the path of light incident on the barcode or a path of light reflected from the barcode so that the ambient light reflected from the barcode is perpendicularly incident on the light detector.

5. The immunodiagnostic fluorescence reader of claim 4,
wherein the light detector comprises an ambient light detector and a fluorescence detector,
wherein the ambient light detector is disposed on the path of the light emitted from the barcode so that the ambient light reflected from the barcode is perpendicularly incident on the ambient light detector.

6. The integrated immunodiagnostic fluorescence reader of claim 3,
wherein the light sources are two,
wherein the path of the light incident on the barcode is different in the ambient light guide part and in the fluorescence guide part,
wherein each light emitted by the two light sources is guided to the detectors along the ambient light guide part and along the fluorescence guide part, respectively,
wherein a light source of the light incident on the ambient light guide part is disposed adjacent to the light detector so that the ambient light reflected from the barcode is perpendicularly incident on the light detector.

7. The immunodiagnostic fluorescence reader of claim 6,
wherein the light detector comprises the ambient light detector and the fluorescence detector,
wherein the light source of the light incident on the ambient light guide part and the ambient light detector are adjacently disposed so that the ambient light reflected from the barcode is perpendicularly incident on the light detector.

8. The integrated immunodiagnostic fluorescence reader of claim 1, wherein the driving module comprises:
a motor configured to provide a rotatory power;
two guide shafts each having one end fixed to the fitting grooves of the base frame and the other end mounted in the mounting grooves, disposed in parallel to the lengthwise direction of the diagnostic cartridge inserted into the inner space, and the two guide shafts configured to guide the optical module by the cylinder barrel and guide protrusions;
a driving shaft connected to the motor in a way to rotate and disposed on at least one lateral side of the optical module, and extending in a front and back direction; and
a driving carrier disposed between the optical module and the driving shaft and connected to the optical module and the driving shaft,
wherein when the motor rotates and thus the driving shaft is rotated, the driving carrier is displaced in the front and back direction, and the optical module connected to the driving carrier is guided by the two guide shafts and displaced in the front and back direction.

9. The integrated immunodiagnostic fluorescence reader of claim 1, wherein the cartridge fixing part of the base frame comprises:
a fixing spring configured to lift up and fix a bottom of the diagnostic cartridge;
a lifting-up protrusion configured to be matched with and fix a portion of the bottom groove of the diagnostic cartridge; and
a side fitting frame configured to fix the diagnostic cartridge by inserting a side of the diagnostic cartridge therein.

10. The integrated immunodiagnostic fluorescence reader of claim 2, wherein the reference position detection module comprises:
a module main board fixed in a direction opposite to the entrance part of the base frame; and
an interrupt sensor part disposed in a direction opposite to the entrance part on the module main board, wherein the interrupt protrusion part is inserted into the interrupt sensor part,
wherein a position of the optical module is displaced between the reference position where the interrupt protrusion part is inserted into the interrupt sensor part and a position of the entrance part.

11. The integrated immunodiagnostic fluorescence reader of claim 10, wherein the reference position detection module further comprises a cartridge sensing switch disposed under the module main board,
wherein the diagnostic cartridge is configured to press the cartridge sensing switch when the diagnostic cartridge is inserted into the base frame through the entrance part and an end of the diagnostic cartridge reaches a specific position, and
wherein the cartridge sensing switch generates an insertion signal for the diagnostic cartridge when the diagnostic cartridge presses the cartridge sensing switch.

12. The integrated immunodiagnostic fluorescence reader of claim 1, wherein the diagnostic cartridge comprises the fluorescence measurement window extended in a lateral flow direction of a sample and having the lateral flow of the sample exposed upward, and a barcode for identifying a type of sample,
wherein the optical module is configured to:
identify the type of sample by reading the barcode by moving to the barcode area,
move to an end point of the fluorescence measurement window after detecting lateral flow start time of the sample at a start point of the fluorescence measurement window, and
calculate a flow rate of the lateral flow by detecting lateral flow end time of the sample at the end point of the fluorescence measurement window.

13. The immunodiagnostic fluorescence reader of claim 6, wherein the diagnostic cartridge comprises the fluorescence measurement window extended in a lateral flow direction of a sample and having the lateral flow of the sample exposed upward, and a barcode,
wherein the optical module is configured to:
identify the type of sample by reading the barcode by moving for the barcode light source to irradiate a light to the barcode;
move to an end point of the fluorescence measurement window after detecting lateral flow start time of the sample at a start point of the fluorescence measurement window, and
calculate a flow rate of the lateral flow by detecting lateral flow end time of the sample at the end point of the fluorescence measurement window.

14. The immunodiagnostic fluorescence reader of claim 7, wherein the diagnostic cartridge comprises the fluorescence measurement window extended in a lateral flow direction of a sample and having the lateral flow of the sample exposed upward, and a barcode,
wherein the optical module is configured to:
identify the type of sample by reading the barcode by moving for the barcode light source to irradiate a light to the barcode;
move to an end point of the fluorescence measurement window after detecting lateral flow start time of the sample at a start point of the fluorescence measurement window, and calculate a flow rate of the lateral flow by detecting lateral flow end time of the sample at the end point of the fluorescence measurement window.
